(19) United States
(12) Patent Application Publication
(10) Pub. No.: US 2011/0054468 A1
(43) Pub. Date: Mar. 3, 2011

(54) Apparatus for Performing an Electrosurgical Procedure

(75) Inventor: Sean T. Dycus, Zurich (CH)

(73) Assignee: TYCO Healthcare Group LP

(21) Appl. No.: 12/551,944

(22) Filed: Sep. 1, 2009

(51) Int. Cl. A61B 18/14

(52) U.S. Cl. 606/45

(57) ABSTRACT

A surgical instrument is provided. The surgical instrument including an end effector assembly operatively connected to a distal end of the shaft and having a pair of first and second jaw members, one or both of the first and second jaw members moveable from an open position to a clamping position, wherein each of the first and second jaw members includes a cutting channel defined therein that extends therethrough. A cutting element is movable within the cutting channel and includes a stationary blade and a pivoting blade that pivots with respect to the stationary blade when first and second jaw members are in the clamped position and the cutting element is advanced through the cutting channel. The jaw members include an actuator operably connected to the housing and configured to impart reciprocating movement of the cutting element.

### BACKGROUND

1. Technical Field

The present disclosure relates to an apparatus for performing an electrosurgical procedure. More particularly, the present disclosure relates to an electrosurgical apparatus that includes a cutting element including a stationary blade and a pivoting blade.

2. Description of Related Art

Electrosurgical instruments (e.g., opened and closed type electrosurgical forceps) are well known in the medical arts and typically include a housing, a handle, one or more shafts and an end effector assembly, which includes jaw members operatively coupled to a distal end of the shaft, that is configured to manipulate tissue (e.g., grasp and seal tissue). Electrosurgical forceps utilize both mechanical clamping action and electrical energy to effect hemostasis by heating the tissue and blood vessels to coagulate, cauterize, seal, cut, desiccate, and/or fulgurate tissue.

Many electrosurgical instruments have been designed to incorporate a cutting or blade element which effectively severs tissue. For example, commonly-owned U.S. application Ser. Nos. 10/116,944 and 10/179,863 describe one such endoscopic instrument which effectively seals and cuts tissue along the tissue seal. Typically, the cutting element is operably associated with the jaw members of the end effector assembly of the electrosurgical forceps.

In some instances, the jaw members may narrow or taper near a distal tip of the jaw members, especially in those instances where the jaw members are configured for small dissection surgical procedures. Due to design constraints associated with end effector assemblies and/or jaw members, the cutting element in certain instances is prevented or impeded from cutting to the distal end of the jaw members.

### SUMMARY

The present disclosure provides a forceps. The forceps includes an end effector assembly having a pair of first and second jaw members. One or both of the first and second jaw members are moveable from an open position wherein the jaw members are disposed in spaced relation relative to one another, to a clamping position wherein the first and second jaw members cooperate to grasp tissue therebetween. In embodiments, each of the first and second jaw members includes a cutting channel defined therein that extends therethrough. A cutting element is movable within the cutting channel, and includes a stationary blade and a pivoting blade, the pivoting blade configured to pivot with respect to the stationary blade when the first and second jaw members are in the clamped position and the cutting element is advanced through the cutting channel. An actuator configured to impart reciprocating movement of the cutting element.

The present disclosure also provides a surgical instrument configured to manipulate tissue. The surgical instrument includes a housing having a shaft that extends therefrom that defines a longitudinal axis therethrough. An end effector assembly is operatively connected to a distal end of the shaft and includes a pair of first and second jaw members. The first and second jaw members are moveable from an open position wherein the jaw members are disposed in spaced relation relative to one another, to a clamping position wherein the first and second jaw members cooperate to grasp tissue therebetween. In embodiments, each of the first and second jaw members includes a cutting channel defined therein that extends therethrough. A handle assembly operatively connects to the housing and includes a movable handle movable relative to a fixed handle operably connected to impart movement of the jaw members relative to each other. A cutting element is movable within the cutting channel, and includes a stationary blade and a pivoting blade, the pivoting blade configured to pivot with respect to the stationary blade when first and second jaw members are in the clamped position and the cutting element is advanced through the cutting channel. An actuator operably connects to the housing and is configured to impart reciprocating movement of the cutting element.

### BRIEF DESCRIPTION OF THE DRAWING

Various embodiments of the present disclosure are described hereinbelow with references to the drawings, wherein:

FIG. 1 is a right, front perspective view of an endoscopic bipolar forceps suitable for use with a cutting element according to an embodiment of the present disclosure;

FIG. 2 is a left, front perspective view of an open bipolar forceps suitable for use with a cutting element according to an embodiment of the present disclosure;

FIG. 3 is an enlarged view of the area of detail illustrated in FIG. 1 with the cutting element in a retracted position and shown within a shaft associated with the bipolar forceps illustrated in FIG. 1;

FIG. 4 is a side view of the cutting element illustrated in FIG. 3 in an advanced position within a pair of jaw members of the end effector assembly associated with the bipolar forceps illustrated in FIG. 1; and

FIG. 5 is a front view of a stationary blade and a pivoting blade of the cutting element illustrated in FIG. 3 with the stationary blade and pivoting blade shown in an initial position.

### DETAILED DESCRIPTION

Detailed embodiments of the present disclosure are disclosed herein; however, the disclosed embodiments are merely exemplary of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In the drawings and in the descriptions that follow, the term “proximal,” as is traditional, will refer to an end of a surgical instrument which is closer to a user, while the term “distal” will refer to an end that is farther from the user. As used herein, the term forceps is meant to include surgical instruments that are intended for use in open or closed surgical procedures including those surgical instruments that are configured for use in bipolar and monopolar modes of operation.

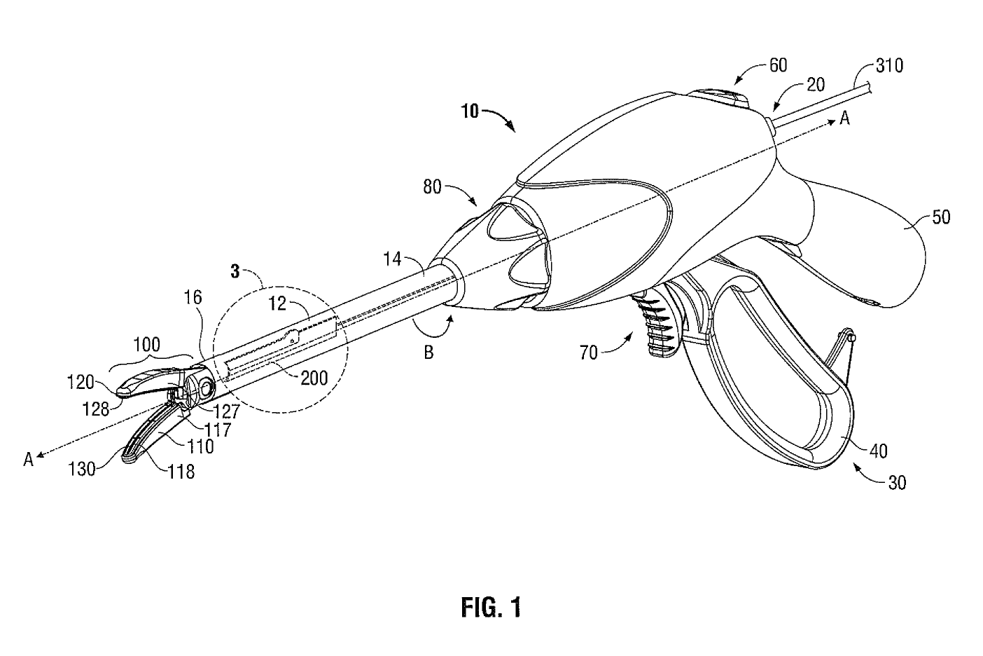

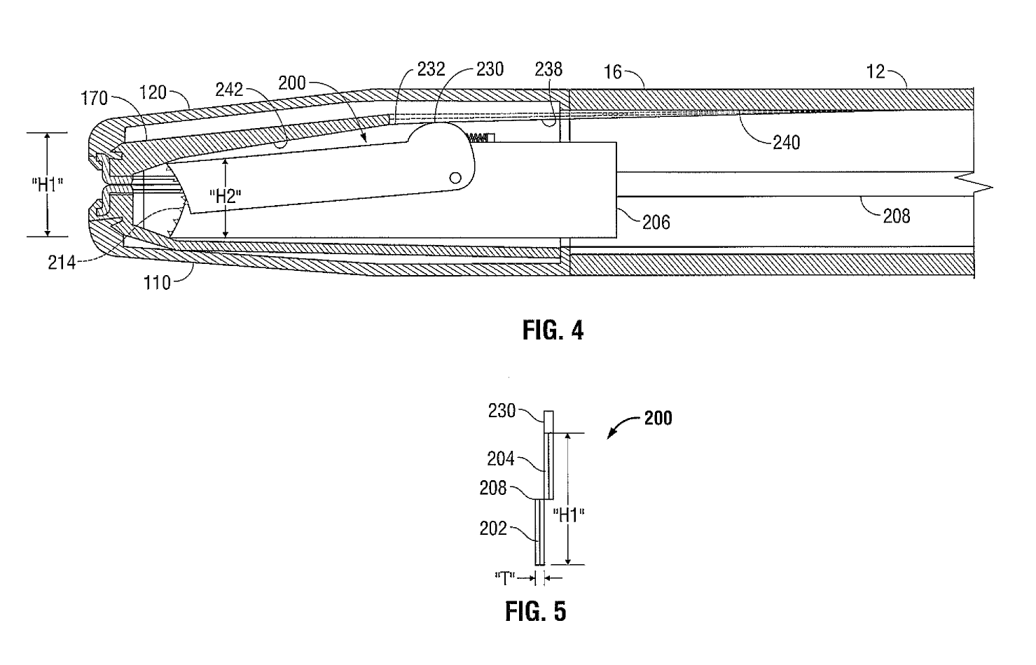

With reference to FIG. 1, an illustrative embodiment of an electrosurgical apparatus (e.g., endoscopic bipolar forceps 10) configured for use with the cutting element 200 according to an embodiment of the present disclosure is shown. Bipolar forceps 10 is shown for use with various electrosurgical procedures and generally includes a housing 20, an electrosurgical cable 310 that connects the forceps 10 to a source of electrosurgical energy (e.g., electrosurgical generator not shown), a handle assembly 30 including a fixed handle 50 and a movable handle 40, a rotating assembly 80, a drive assembly (not shown), an end effector assembly 100 that operatively connects to the drive assembly. The drive assembly may be in operative communication with handle assembly 30 for imparting movement of one or both of a pair of jaw members 110, 120 of end effector assembly 100. End effector assembly 100 includes opposing jaw members 110 and 120 that are operatively and pivotably coupled to each other and fixedly attached to a distal end 16 of a shaft 12 (FIG. 1). In certain embodiments, each of the jaw members 110 and 120 are pivotable with respect to each other (i.e., a bilateral jaw configuration). In certain embodiments, one of the jaw members, e.g., jaw member 110, is pivotable with respect to the other jaw member, which is stationary, e.g., jaw member 120 (i.e., a unilateral jaw configuration). Jaw members 110, 120 mutually cooperate to grasp, seal and, in some cases, divide large tubular vessels and large vascular tissues. Each of the first and second jaw members 110 and 120, respectively, includes a tapered distal end, the two tapered distal ends forming a tapered height when the jaw members are clamped in the closed position (FIG. 4). Forceps 10 includes an actuator or a trigger assembly 70 operably coupled to the housing 20 and configured to impart reciprocating movement of the cutting element 200 through a channel 130 defined within the jaw members 10 and 120. A proximal end 14 of the shaft 12 is configured to mechanically engage the housing 20.

Jaw member 110 includes an insulative jaw housing 117 and an electrically conductive seal plate 118. The insulative housing 117 is configured to securely engage the electrically conductive seal plate 118. This may be accomplished by stamping, by overmolding, by overmolding a stamped electrically conductive sealing plate and/or by overmolding a metal injection molded seal plate. All of these manufacturing techniques produce an electrode having a seal plate 118 that is substantially surrounded by the insulating substrate. Within the purview of the present disclosure, jaw member 110 may include a jaw housing 117 that is integrally formed with a seal plate 118.

Jaw member 120 includes a similar structure having an outer insulative housing 127 that may be overmolded to capture seal plate 128.

For a more detailed description of the bipolar forceps 10 including end effector assembly 100, handle assembly 30 including movable handle 40, trigger assembly 70, electrosurgical cable 310 (including line-feed configurations and/or connections) and other operative components associated with the forceps 10, reference is made to commonly owned United States Patent Publication No. 2003/0229344 and U.S. Pat. No. 7,150,749.

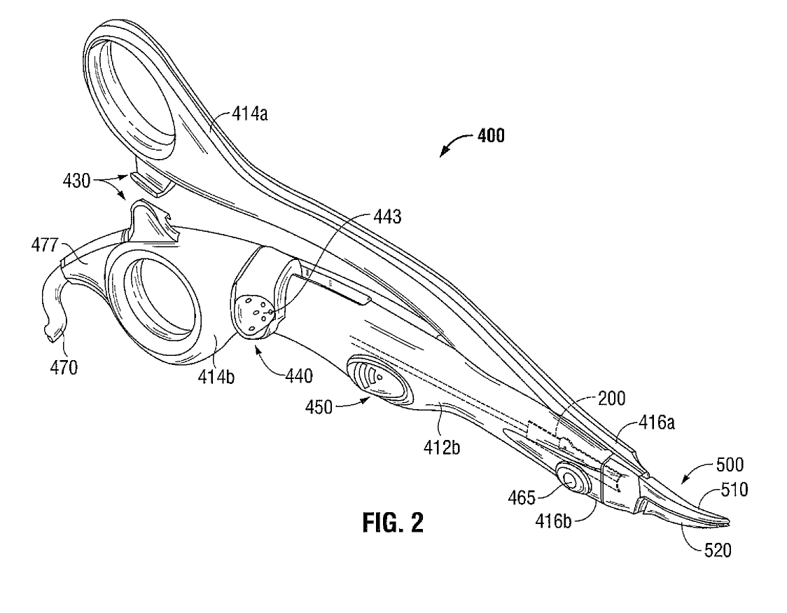

With reference to FIG. 2, an illustrative embodiment of an electrosurgical apparatus (e.g., open bipolar forceps 400) configured for use with the cutting element 200 is shown. Forceps 400 is configured for use with open surgical procedures and includes elongated shaft portions 412a and 412b each having a proximal end 414a, 414b and a distal end 416a and 416b, respectively. Forceps 400 includes an end effector assembly 500 that attaches to the distal ends 416a and 416b of shafts 412a and 412b, respectively. Shaft 412b may be generally hollow to house a handswitch 450 (and the electrical components associated therewith). A proximal shaft connector 477 electromechanically engages an electrosurgical cable 470 such that a user may selectively apply electrosurgical energy as needed. More particularly, a handswitch 450 is configured to permit a user to selectively apply electrosurgical energy as needed to seal tissue grasped between jaw members 510 and 520.

A ratchet mechanism 430 is disposed at the proximal ends 414a, 414b of shafts 412a, 412b, respectively, for selectively locking the jaw members 510 and 520 relative to one another in one or more positions during pivoting. The end effector assembly 500 includes pair of opposing jaw members 510 and 520 that are pivotably connected about a pivot assembly 465 and which are movable relative to one another to grasp tissue. Forceps 400 includes an actuating mechanism 440 to advance the cutting element 200. More particularly, the actuating mechanism 440 includes a trigger or finger tab 443 that is operatively associated with a first gear rack (not shown) such that movement of the trigger or finger tab 43 moves the first rack in a corresponding direction. The actuating mechanism 440 mechanically cooperates with a second gear rack (not shown) that is operatively associated with a drive rod (not shown) and which advances the entire cutting mechanism 200.

For a more detailed description of the forceps 400 including end effector 500, actuation member 440 and other operative components associated with the forceps 400, reference is made to commonly owned United States Patent Publication No. 2005/0154387.

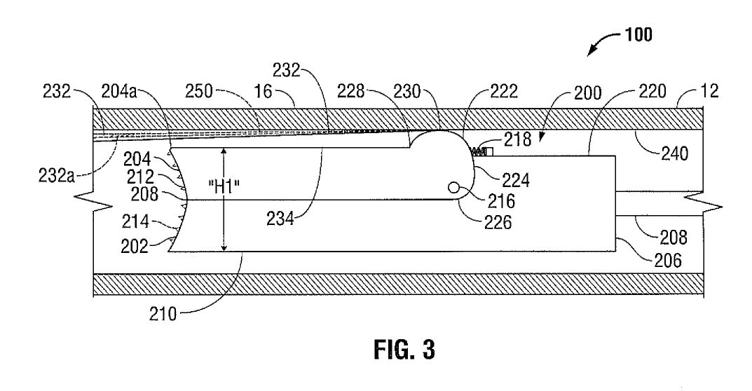

With reference to now to FIGS. 3-5, and initially with reference to FIG. 3, an embodiment of a cutting element 200 configured for use with either forceps 10 or 400 is shown. For the purposes of brevity, hereinafter the cutting element 200 and operative components associated therewith will be described in terms of use with forceps 10.

Cutting element 200 and operative components associated therewith may be formed from any suitable material, e.g., biocompatible grade steel. Cutting element 200 may be formed by any known methods including but not limited to stamping, machining, spot welding, and the like. Cutting element 200 is operably connected to and in mechanical communication with trigger 70. Trigger 70 and operative components associated therewith are configured to impart reciprocating movement of the cutting element 200 through a channel 130 (FIG. 1). In the embodiment illustrated in FIG. 1, each of the first and second jaw members define the channel 130. Cutting element 200 includes a proximal end 206 that operably couples to a connector 208 (as best seen in FIG. 4) or other suitable device that is in operative communication with the trigger 70. In an embodiment, the proximal end 206 may operably couple to a push rod (not shown) that couples to an actuator that is movable from a proximal to distal position, such as, for example, the actuator mechanism 440 illustrated in FIG. 2, or other suitable actuating mechanisms, such as, for example a drive wheel that translates the push rod forward when the drive wheel is rotated. Cutting element includes a distal end 210 that includes a cutting edge 212 that is defined by a stationary blade 202 and a pivoting blade 204. In the embodiment illustrated in FIG. 3, cutting edge 212 includes a generally arcuate or concave configuration. It is within the purview of the present disclosure, that cutting edge 212 may include a generally convex or straight configuration. Cutting element 200 (or portion thereof) includes a first height “H₁” when the cutting element 200 is in a retracted position and the stationary blade 202 and pivoting blade 204 are in a “non-folded” configuration (FIGS. 3 and 5). Cutting element (or portion thereof) also includes a second height “H₂” when the cutting element 200 is in an advanced position and the stationary blade 202 and pivoting blade 204 are in a “folded” configuration (see FIG. 4).

With reference again to FIG. 3, cutting element 200 includes stationary blade 202 and pivoting blade 204. In some embodiments, the stationary blade 202 and pivoting blade 204 are interleaved. Pivoting blade 204 pivots with respect to the stationary blade 202 when the first and second jaw members 110 and 120, respectively, are in the clamped position and the cutting element 200 is advanced through the channel 130 towards a distal end of the first and second jaw members 110 and 120, respectively (FIG. 4).

Stationary blade 202 is formed at the distal end 210 of cutting element 200. In the embodiment illustrated in FIGS, 1-4, stationary blade 202 is positioned lower than, and in an offset relation relative to, the pivoting blade 204, as best seen in FIG. 5.

As can be appreciated, this blade configuration allows the cutting element 200 to advance through the cutting channel 130 to the distal end of the jaw members 110, 120 by virtue of the pivoting blade 204 being cammed to follow the tapered profile of the cutting channel 130. In other words, as the cutting element 200 advances through the cutting channel 130, the pivoting blade 204 of the cutting element 200 is cammed to reduce the height (from “H₁” to “H₂”) thereof to match the profile of the tapered jaw members 110 and 120, thereby allowing the cutting element 200 to advance further along the cutting path to sever tissue.

This configuration of a stationary blade 202 that is positioned lower than and in an offset relation relative to the pivoting blade 204 may also provide a scissor-like cutting action between the stationary blade 202 and pivoting blade 204, wherein the scissor-like cutting action facilitates severing of tissue when the cutting element 200 is translated through the channel 130, especially after a tissue sealing procedure.

A top leading edge 204a of the pivoting blade 204 includes a generally rounded or curved configuration (as best seen in FIG. 3). This configuration of a top leading edge 204a that is rounded, as opposed to a leading edge that is straight or flat, facilitates movement and/or pivoting of the pivoting blade 204 as the cutting element 200 is advanced through the cutting channel 130. More particularly, the rounded configuration of the top leading edge 204a minimizes or reduces the chances of the top leading edge 204a cutting into an interior wall of the shaft 12 and/or jaw member 120. Alternatively, a top leading edge 204a may be relatively straight of flat (as best seen in FIG. 4). In an embodiment, stationary blade 202 and pivoting blade 204 are offset from each other a distance that is substantially equal to the thickness “T” of the stationary blade 202, see FIG. 5 for example.

Pivoting blade 204 is pivotably coupled to cutting element 200. Pivoting element may be pivotably coupled to cutting element 200 by any suitable pivoting means, such as, for example, a living hinge, a pivot pin, etc. In the embodiment illustrated in FIG. 3, pivoting blade 204 is pivotably coupled to stationary blade 202 via a pivoting pin 216 operatively disposed adjacent proximal end 206 of cutting element 200. Pivoting pin 216 is configured to provide a point of pivot for the pivoting blade 204 when the cutting element 200 is moved from the retracted position to the advanced position, and vice-versa.

In an embodiment, a spring or other suitable biasing element 218 (e.g., elastic band or the like) is operatively disposed on the cutting element 200 and operatively couples the cutting element 200 to a proximal end 222 of pivoting blade 204. Spring 218 is configured to bias the pivoting blade 204 in an upright, or non-pivoted or unfolded position when the cutting element 200 is in the retracted position or when the cutting element is being moved from the advanced position to the retracted position. In the embodiment illustrated in FIG. 3, spring 218 is operatively disposed at a predetermined position on a top portion 220 of the cutting element 200. The proximal end 222 may include a generally arcuate portion 224 that extends from a bottom portion 226 of the pivoting blade 204 to a top portion 228 of the pivoting blade 204.

Pivoting blade 204 includes one or more camming structures that may be in the form of one or more protuberances or nibs 230. Protuberance 230 is configured to contact one or more camming surfaces 232 (to be described in more detail below) such that as cutting element 200 is advanced through the channel 130, the pivoting blade 230 is caused to pivot or deflect downward toward the stationary blade 202 to reduce the overall height of the cutting element 200 and to promote a scissor-like inter-action between the stationary blade 202 and pivoting blade 204 (as noted above this scissor-like action may facilitate severing tissue). With this purpose in mind, protuberance 230 is located at a predetermined position along a top surface 234 of pivoting blade 204 at a point that is distal the pivot pin 216. This configuration of a protuberance 230 that is located distal a pivot pin 216 facilitates pivoting the pivoting blade 204 when the protuberance 230 contacts the camming surface 232. That is, because the protuberance 230 is located distal the pivot pin 216, the pivoting blade 204 is prevented from pivoting upward or toward a top portion of the shaft 12 and/or jaw member, e.g., jaw member 110, when the protuberance contacts the camming surface 232. The dimensions, e.g. the size, of protuberance 230 may depend on a number of factors, such as, for example, the desired rate of deflection of the pivoting blade 204 with respect to the velocity of the cutting element 200 through the channel 130 during actuation the trigger assembly 70.

With continued reference to FIG. 3, camming surface 232 is shown. As noted above, camming surface 232 is configured to contact or cam protuberance 230 of the pivoting blade 204 such that pivoting blade 204 is caused to pivot or deflect downward towards the stationary blade 202 when the cutting element 200 is advanced distally through the channel 130. To this end, camming surface 232 may be operatively disposed along an upper portion of the distal end of the shaft 12 and/or jaw member, e.g., jaw member 110.

In the embodiments illustrated in FIGS. 1-5, camming surface 232 is located at each of the distal end 16 of the shaft 12 and a proximal end 238 of the jaw member 120. More particularly, camming surface 232 extends along a top inner surface 240 of the distal end 16 of shaft 12 (FIG. 3) to a top inner surface 242 of the proximal end of the jaw member 110. Camming surface 232 includes a continuous, uniform slanted or sloped (e.g., ramp-like) configuration that includes a rate of inclination that, together with the size of protuberance 230, provides an even, uniform camming of the pivoting blade 204 when the pivoting blade 204 is moved from the retracted to advanced position and vice-versa.

Alternatively, camming surface 232 may include a plurality of non-continuous or intermittent, camming surfaces 232a (shown phantomly in FIG. 3) that, together with the size of protuberance 230, may provide a non-uniform camming of the pivoting blade 204 when the pivoting blade 204 is moved from the retracted to advanced position and vice-versa. In either embodiment, after the cutting element 200 has fully advanced through the channel 130, cutting element 200 or portion thereof will have a height “H₂”, as described hereinabove. In the instance, where a non-continuous camming surface 232a is implemented, after the cutting element 200 has been advanced, a final camming surface (not shown) of the plurality of camming surfaces 232a may be configured to cause the pivoting blade 204 to deflect downward a distance that provides cutting element 200 with a height “H₂”. The exact dimension or configuration of either of the camming surfaces 232 and 232a will depend on the contemplated uses of a manufacturer.

Operatively disposed on along the top inner surface 240 of the distal end 16 of the shaft 12 and/or the jaw member, e.g., jaw member 110, may be one or more grooves or slots 250 configured to receive cutting element 200 or portion thereof In the embodiment illustrated in FIG. 3, slot 250 is located at each of the distal end 16 of the shaft 12 and a proximal end 238 of the jaw member 120. More particularly, slot 250 extends along the top inner surface 240 of the distal end 216 of shaft 12 (FIG. 3) to the top inner surface 242 of the proximal end of the jaw member 120 within the camming surface 232. Alternatively, slot 250 may be located adjacent camming surface 232.

Prior to cutting element 200 being actuated, cutting element 200 is in an initial retracted position and has a first height “H₁”. After tissue has been properly grasped and electrosurgically treated, e.g., sealed, a user may actuate or squeeze trigger 70. Actuation of trigger 70 causes cutting element 200 to move from the initial retracted position (FIG. 3) to a final advanced position. During translation of cutting element 200 through channel 130, camming surface 232 contacts protuberance 230 of pivoting blade 204 and causes pivoting blade 204 to pivot and/or deflect downward toward stationary blade 202 (FIG. 4). In the embodiment where a continuous camming surface 232 is employed, cutting element 200 severs tissue in an even, uniform fashion, i.e. scissor-like manner. In the embodiment where a non-continuous camming surface 232a is employed, cutting element 200 severs tissue in generally saw-tooth fashion. During the translation of pivoting blade 204 through channel 130, the height of cutting element 200 transitions from a height “H₁” where the cutting element 200 is in the retracted position to a height “H₂” where the cutting element 200 is in the advanced position. Thus, in the instance where a distal end 170 of the jaw members narrow or taper, the cutting element 200 is not impeded or prevented from advancing to the distal end 170 of the jaw members. After tissue has been severed, actuation trigger 70 may be actuated again or released. In an embodiment, releasing trigger 70 causes cutting element to translate proximally through channel 130 and back toward the retracted position. As cutting element 200 translates proximally, pivoting blade 204 is caused to return to the initial position of the pivoting blade 204 under the bias of spring 218.

From the foregoing and with reference to the various figure drawings, those skilled in the art will appreciate that certain modifications can also be made to the present disclosure without departing from the scope of the same. For example, in an embodiment it may prove advantageous to have one of the stationary blade 202 and pivoting blade 204 include one or more serrations 214 (shown phantomly in FIG. 3). In this instance, the serrations 214 are intended facilitate severing tissue when the cutting element 200 is advanced through the channel 130. Additionally, it may prove advantageous for the stationary blade 202 and pivoting blade 204 to have different dimensions. For example, pivoting blade 204 may include a depth, width, and/or height that is different from that of stationary blade 202. As can be appreciated by one skilled in the art, the exact dimension and/or configuration of the stationary blade 202 and pivoting blade 204 with respect to each other will depend on the contemplated uses of a manufacturer.

In some embodiments, it may prove advantageous to have one or both of the stationary and pivoting blades 202 and 204, respectively, define a cutting edge 212 that includes a bevel, facet, etc.

In some embodiments, a portion of the cutting element 200 may include one or more cam slots or grooves that are configured to receive one or more camming structures operatively disposed on the pivoting blade 204. More particularly, an inner side surface of the cutting element 200 may include a cam slot that is configured to receive a corresponding camming structure, e.g., a cam pin disposed on an inner side surface of the pivoting blade 204. In this instance, the cam slot may include a contour commensurate with the cam pin and may be configured to provide a path of motion for the pivoting blade 204. In this instance, the cam slot may include a generally arcuate configuration with all points equidistant from the center of the cam pin. This combination of cam slot and cam pin may further facilitate severing tissue when the cutting element 200 is advanced through the channel 130. As can be appreciated by one skilled in the art, the cam pin 216 and/or pivoting blade 204 may be configured to compensate for the path of motion of the cam pin. The cam slot and cam pin may be formed on the respective inner surfaces of the cutting element 200 and pivoting blade 204 may be formed by any suitable means, such as, for example, by known etching techniques.

While the above-referenced cutting element of the present disclosure has been described herein in terms of a cutting element 200 that includes a pivoting blade 204 that is disposed offset and above the stationary blade 202, it is within the purview of the present disclosure to have a pivoting blade 204 that is offset and below the stationary blade 202. As can be appreciated by one skilled in the art, in this instance certain design modifications will need to be implemented in the manufacturing process of the cutting element 200 and/or bipolar forceps 10 and the pivoting blade 204 will be configured to pivot upwards.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A forceps, comprising:
an end effector assembly having a pair of first and second jaw members, at least one of the first and second jaw members moveable from an open position wherein the jaw members are disposed in spaced relation relative to one another, to a clamping position wherein the first and second jaw members cooperate to grasp tissue therebetween, wherein each of the first and second jaw members includes a cutting channel defined therein that extends therethrough;
a cutting element movable within the cutting channel, the cutting element including a stationary blade and a pivoting blade, the pivoting blade configured to pivot with respect to the stationary blade when the first and second jaw members are in the clamped position and the cutting element is advanced through the cutting channel; and
an actuator configured to impart reciprocating movement of the cutting element.

2. The forceps of claim 1, wherein the stationary blade and the pivoting blade are pivotably coupled to each other via a pivot pin located at a proximal end of the cutting element.

3. The forceps of claim 1, wherein the stationary blade and pivoting blade are offset from each other and configured such that as the cutting element is advanced through the cutting channel, the pivoting blade moves in a scissor-like fashion relative to the stationary blade to facilitate severing of tissue.

4. The forceps of claim 1, wherein at least one of the stationary blade and pivoting blade includes a serrated cutting edge.

5. The forceps of claim 1, wherein a groove is operably disposed on at least one of the first and second jaw members and configured to receive at least a portion of the pivoting blade.

6. The forceps of claim 5, wherein an upper portion of the pivoting blade includes a tab located distally relative to the pivot pin that is configured to translate within the groove.

7. The forceps of claim 6, wherein the groove includes a camming surface configured to cam the tab when the cutting element is being advanced through the cutting channel.

8. The forceps of claim 7, wherein the cutting element has a first height when the stationary blade and pivoting blade are in a retracted position and a second height when the stationary blade and pivoting blade are in fully advanced position, wherein the second height is less than the first height.

9. The forceps of claim 1, wherein the cutting element further includes a biasing element configured to bias the pivoting blade in an initial non-pivoted condition relative to the stationary blade when the cutting element is in the retracted position.

10. The forceps of claim 1, wherein the stationary blade and pivoting blade each include a thickness “T”, wherein the stationary blade and pivoting blade are offset from each other a distance that is about equal to the thickness “T”.

11. The forceps of claim 1, wherein the stationary blade and pivoting blade are interleaved.

12. A surgical instrument, comprising:
a housing having a shaft that extends therefrom that defines a longitudinal axis therethrough;
an end effector assembly operatively connected to a distal end of the shaft and having a pair of first and second jaw members, at least one of the first and second jaw members moveable from an open position wherein the jaw members are disposed in spaced relation relative to one another, to a clamping position wherein the first and second jaw members cooperate to grasp tissue therebetween, wherein the each of the first and second jaw members includes a cutting channel defined therein that extends therethrough;
a handle assembly including a movable handle movable relative to a fixed handle operably connected to impart movement of the jaw members relative to each other;
a cutting element movable within the cutting channel, the cutting element including a stationary blade and a pivoting blade, the pivoting blade configured to pivot with respect to the stationary blade when the first and second jaw members are in the clamped position and the cutting element is advanced through the cutting channel; and
an actuator operably connected to the housing and configured to impart reciprocating movement of the cutting element.

13. The surgical instrument of claim 12, wherein the stationary blade and the pivoting blade are pivotably coupled to each other via a pivot pin located at a proximal end of the cutting element.

14. The surgical instrument of claim 12, wherein the stationary blade and pivoting blade are offset from each other and configured such that as the cutting element is advanced through the cutting channel, the pivoting blade moves in a scissor-like fashion relative to the stationary blade to facilitate severing of tissue.

15. The surgical instrument of claim 12, wherein a groove is operably disposed on at least one of the first and second jaw members and is configured to receive at least a portion of the pivoting blade.

16. The surgical instrument of claim 12, wherein an upper portion of the pivoting blade includes a tab located distally relative to the pivot pin that is configured to translate within the groove.

17. The surgical instrument of claim 16, wherein the groove includes a camming surface configured to cam the tab when the cutting element is being advanced through the cutting channel.

18. The surgical instrument of claim 17, wherein the cutting element has a first height when the stationary blade and pivoting blade are in a retracted position and a second height when the stationary blade and pivoting blade are in fully advanced position, wherein the second height is less than the first height.

19. The surgical instrument of claim 12, wherein the cutting element further includes a biasing element configured to bias the pivoting blade in an initial non-pivoted condition relative to the stationary blade when the cutting element is in the retracted position.

20. The surgical instrument of claim 12, wherein the stationary blade and pivoting blade each include a thickness “T”, wherein the stationary blade and pivoting blade are offset from each other a distance that is about equal to the thickness “T”.
